(19) United States
(12) Patent Application Publication
(10) Pub. No.: US 2025/0163283 A1
(43) Pub. Date: May 22, 2025

(54) SYSTEM AND PROCESS FOR PRINTING ENCAPSULATED AND NON-ENCAPSULATED MATERIALS UTILIZING GELLING AGENTS

(71) Applicant: Temptime Corporation, Morris Plains, NJ (US)

(72) Inventors: Brian S. HUFFMAN, Belle Mead, NJ (US); Thi N. DO, West Orange, NJ (US)

(73) Assignee: Temptime Corporation, Morris Plains, NJ (US)

(21) Appl. No.: 18/516,510

(22) Filed: Nov. 21, 2023

(51) Int. Cl. C09D 11/033; C09D 11/50; G01J 5/58; G01K 11/12; G01N 33/00

(52) U.S. Cl. CPC C09D 11/033; C09D 11/50; G01J 5/58; G01K 11/12; G01N 33/0036

(57) ABSTRACT

Disclosed is a media element, comprising a media substrate, a gelled material adhered to the media substrate, and a plurality of microcapsules suspended in the gelled material. Each microcapsule encapsulating an environmental indicator material in a non-activated configuration and releasing the environmental indicator material when ruptured, the environmental indicator material configured, in response to exposure to a predetermined environmental condition, to change from an initial state to a second state producing an observable effect. The plurality of microcapsules prevent an occurrence of the observable effect while in the non-activated configuration and allow the occurrence of the observable effect when the environmental indicator material is exposed to the predetermined environmental condition after at least a portion of the plurality of microcapsules are ruptured.

### BACKGROUND

The present disclosure generally relates to printing on media, such as labels and/or tags that can be printed or otherwise processed with media processing devices, such as printers. Once the media is processed by the media processing device, the media can be placed on objects, such as packages, products for sale at retail, stock shelf labels, etc. A supply of media may be provided in various forms, such as a roll or stack, and the media may come in different sizes (e.g., different shapes, lengths and/or widths).

### BRIEF DESCRIPTION OF THE SEVERAL VIEWS OF THE DRAWINGS

The novel features of the various aspects are set forth with particularity in the appended claims. Throughout the FIGS. like reference characters designate like or corresponding parts throughout the several views of the drawings. The described aspects, however, both as to organization and methods of operation, may be best understood by reference to the following description, taken in conjunction with the accompanying drawings in which:

FIG. 1 illustrates an example view of microcapsules of an environmental sensor on a media element, where the environmental sensor is in the activated configuration, according to at least one aspect of the present disclosure.

FIG. 2 illustrates a diagram showing a media element exposed to the predetermined environmental condition before and after activation, according to at least one aspect of the present disclosure.

FIG. 3 illustrates an example view of a plurality of microcapsules of an environmental sensor on a media element, according to at least one aspect of the present disclosure.

FIG. 4 illustrates a microcapsule rupturing after an activation process, according to at least one aspect of the present disclosure.

FIGS. 5 and 6 illustrate an example method of making an ink composition with a plurality of microcapsules for screen printing, according to at least one aspect of the present disclosure.

FIG. 7 is a perspective view of a rotary screen printing system comprising a plurality of rotary screen printing devices, according to at least one aspect of the present disclosure.

FIG. 8 is a cross-sectional view of one of the rotary screen printing devices of the plurality of rotary screen printing devices of FIG. 7, according to at least one aspect of the present disclosure.

FIG. 9 is a sectional and detailed view of the rotary screen printing device of FIG. 8, according to at least one aspect of the present disclosure.

FIG. 10 is a sectional view of an alternative rotary screen printing configuration compared to the rotary screen printing device of FIGS. 8 and 9, according to at least one aspect of the present disclosure.

FIG. 11 illustrates an example method of screen printing an ink composition with a plurality of microcapsules, according to at least one aspect of the present disclosure.

FIG. 12 illustrates a diagram of a cross-section of an example media element including an environmental sensor, according to at least one aspect of the present disclosure.

FIG. 13 illustrates a diagram of a cross-section of an example media element including an environmental sensor, according to at least one aspect of the present disclosure.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The following detailed description of example implementations refers to the accompanying drawings. The same reference numbers in different drawings may identify the same or similar elements.

Environmental sensors can be used (e.g. in media elements) to produce an observable effect in response to exposure to a predetermined environmental condition. Some example, observable effects are a change in color state (e.g., hue, transparency, darkness, etc.), a change in an electrical property, or a change a state of matter (e.g., melting or other liquefaction, solidifying) or changes in viscosity or fluidity of an indicator material, in response to exposure to the predetermined environmental condition. As one example, an environmental sensor that uses chemical based indicators may be configured to produce an observable effect when a temperature of the environment is above a predetermined temperature threshold. In some aspects, an environmental sensor requires activation (e.g. by applying a force) before the observable effect can be produced by exposure to the predetermined environmental condition. If the environmental sensor is not activated, then the observable effect is not produced by the exposure to the predetermined environmental condition. This type of environmental sensor is considered an activatable environmental sensor.

An example activatable environmental sensor uses chemical-based indicators which may be selectively activated at the end point of application. This process provides the benefit of allowing the environmental sensor (before activation) to be exposed to the predetermined environmental condition without producing the observable effect. The environmental sensor has a non-activated configuration where the media with the environmental sensor can be stored in the non-activated configuration without worrying about ruining the environmental sensor. For example, if the environmental sensor was designed to respond to a temperature above a response temperature, the media with the environmental sensor can be stored in the non-activated configuration at a temperature above the response temperature without ruining the environmental sensor. This avoids the need to maintain a strict cold chain for the temperature exposure sensors before the sensors are paired with products.

Some activatable environmental sensors rely on keeping indicator materials separate mechanically and then joining them together, e.g., by removing a barrier sheet or bringing two halves a clamshell structure into close contact. Prior to activation, the indicator does not react with relevant environmental stimulus. Another approach is to encapsulate the environmental indicator materials and activate them by rupturing the encapsulation (e.g. by a force) to release the indicator materials as described herein and in commonly owned and related applications: U.S. patent application, titled “PRINTER ACTIVATABLE ENVIRONMENTAL SENSING THROUGH CHEMICAL ENCAPSULATION”, Attorney Docket No. 0820887.00357, filed Sep. 18, 2023; U.S. patent application, titled “MEDIA PROCESSING DEVICE AND COMPONENTS FOR ACTIVATABLE MEDIA PLATFORMS”, Attorney Docket No. 0820887.00358, filed Sep. 18, 2023; U.S. patent application, titled “USE OF ENCAPSULATED POLAR PROTIC CHEMISTRIES FOR RFID TEMPERATURE MONITORING”, Attorney Docket No. 0820887.00355, filed Sep. 18, 2023; U.S. patent application, titled “MEDIA CONSTRUCTION TO FACILITATE USE OF ACTIVATABLE PLATFORM”, Attorney Docket No. 0820887.00359, filed Sep. 18, 2023; and U.S. patent application, titled “RIBBON FOR USE IN PRODUCING PRINTER ACTIVATABLE INDICATORS”, Attorney Docket No. 0820887.00356, filed Sep. 18, 2023.

The encapsulated environmental indicator materials can be placed on media to form an activatable environmental sensor on the media. The environmental sensor can then be later activated with physical forces such as pressure, sheer, cutting or friction sufficient to rupture the encapsulation. The forces can be applied with a device, e.g. a media processing device, or by a user pinching environmental sensor by hand. In some aspects, heat is also applied to the environmental sensor for activation. In at least one aspect, the media can include a bursting additive (e.g. roughness and/or abrasiveness) to increase the ease of rupturing the indicator materials' encapsulation and activate the environmental sensor.

The activable environmental sensors can be manufactured using various printing and dispensing techniques (e.g. rotary screen, flexo, gravure, etc.). However, conventional formulation techniques create issues when trying to print encapsulated materials for a variety of reasons. For example, typically binders and formulating agents are added to create a mixture with the microcapsules to achieve a printable viscosity. However, these formulating components may negatively impact the shell material used to encapsulate the indicator material. The formulating components may also negatively interfere with the ability of the encapsulated indicator material to properly function after activation (i.e., release from the encapsulation shell).

One solution is to use a gelled material to hold the microcapsules to a media element. For example, an ink composition including a gelling agent, a carrier liquid, and a plurality of microcapsules can be used in place of binder, formulating agents, solvents, etc. to achieve a printable ink suitable for delivering activable encapsulated materials onto a media element. In at least one aspect, the ink composition is screen printed to the media element, where the ink composition has a first viscosity during printing and a second viscosity greater than the first viscosity after printing. In at least one aspect, the ink composition is a gelled material that encompasses the microcapsules on the media element.

Using a gelled material for printing has many benefits. For example, an ink composition with a gelling agent can be more suitable for printing than non-thickening or non-gelling liquids since it increases the viscosity of the fluid, making the rheology more suitable for printing processes. Another example, is that the gelling agent can be used to provide a desired thickness and/or viscosity for printing. Additionally, there is no need to eliminate solvents through drying and as a result a more environmentally friendly/green printing process is achieved. The ink composition with a gelling agent significantly reduces the negative impact associated with the use of binders, formulating components, solvent, etc. to deliver activatable encapsulated materials. This approach can also be used to deliver non-encapsulated materials utilizing gelling agents in an ink composition.

In one general aspect, the present disclosure provides a media element, including a media substrate, a gelled material adhered to the media substrate, and a plurality of microcapsules suspended in the gelled material. Each microcapsule encapsulating an environmental indicator material in a non-activated configuration and releasing the environmental indicator material when ruptured, the environmental indicator material configured, in response to exposure to a predetermined environmental condition, to change from an initial state to a second state producing an observable effect. The plurality of microcapsules prevent an occurrence of the observable effect while in the non-activated configuration and allow the occurrence of the observable effect when the environmental indicator material is exposed to the predetermined environmental condition after at least a portion of the plurality of microcapsules are ruptured.

In at least one aspect, the gelled material includes a network expanded throughout a carrier liquid.

In at least one aspect, the carrier liquid includes an organic solvent.

In at least one aspect, the organic solvent includes a polar protic organic solvent.

In at least one aspect, the polar protic organic solvent includes a hydroxyl group.

In at least one aspect, the polar protic organic solvent is chosen from a list including polyethylene glycol, methanol, ethanol, propanol, butanol, pentadecanol, hexanol, decanol, undecanol, dodecanol, and tridecanol.

In at least one aspect, the gelled material comprises a gelling agent comprising an amide.

In at least one aspect, the amide includes Dibutyl Ethylhexanoyl Glutamide, Dibutyl Lauroyl Glutamide, or combinations thereof.

In at least one aspect, the gelled material includes a viscosity of greater than or equal to 10,000 cps at a temperature below 50° C.

In at least one aspect, a viscosity of the gelled material decreases as a temperature of the gelled material is increased.

In at least one aspect, the environmental indicator material flows through the gelled material when released.

In at least one aspect, the observable effect includes at least one effect selected from the group consisting of a color state change, a change in transparency, a change in hue, a change in an electrical property, a change in conductivity, a change in capacitance, a movement of the environmental indicator material along the media substrate, and combinations thereof.

In at least one aspect, the predetermined environmental condition includes at least one condition selected from the group consisting of temperature excursion above a predetermined temperature threshold for at least a predetermined amount of time, temperature excursion below a predetermined temperature for at least a predetermined amount of time, cumulative exposure to temperature over a time period above a predetermined threshold for at least a predetermined amount of time, exposure to a particular chemical, oxygen exposure, ammonia exposure, exposure to a particular chemical above a threshold concentration, exposure to a particular chemical above the threshold concentration for at least a predetermined amount of time, exposure to at least a predetermined amount of radiation of a particular type, ultraviolet light exposure, humidity exposure, exposure to a humidity level above a predetermined threshold, and exposure to a humidity level above a predetermined threshold for at least a predetermined amount of time.

In at least one aspect, at least a portion of the plurality of microcapsules are ruptured based on a force greater than a predetermined threshold, the force being at least one of a pressure force, a shear force, a frictional force, or a cutting force applied to the media element.

In at least one aspect, the microcapsules include a diameter length between 20 to 250 μm.

In another general aspect, the present disclosure provides an ink composition for screen printing. The ink composition includes a carrier composition including a gelling agent and a carrier liquid. The ink composition further includes a plurality of microcapsules. Each microcapsule encapsulating an environmental indicator material in a non-activated configuration and releasing the environmental indicator material when ruptured, the environmental indicator material configured, in response to exposure to a predetermined environmental condition, to change from an initial state to a second state producing an observable effect. The plurality of microcapsules prevent an occurrence of the observable effect while in the non-activated configuration and allow the occurrence of the observable effect when the environmental indicator material is exposed to the predetermined environmental condition after at least a portion of the plurality of microcapsules are ruptured.

In at least one aspect, at a temperature below 50° C., the carrier composition forms a gelled material comprising a network expanded throughout the carrier liquid.

In at least one aspect, the carrier composition includes a viscosity of greater than or equal to 10,000 cps at a temperature below 50° C.

In at least one aspect, a viscosity of the carrier composition decreases as a temperature of the carrier composition increases.

In at least one aspect, the carrier composition includes a first viscosity range associated with a first temperature range and a second viscosity range associated with a second temperature range, the values in the first temperature range are greater than values in the second temperature range, and the second viscosity range is lower than the first viscosity range.

In at least one aspect, the first temperature range is from 50° C. to 80° C. and the second temperature range is less than 50° C.

In at least one aspect, the first viscosity range is from 1000 cps to 10,000 cps and the second viscosity range is greater than 10,000 cps.

In at least one aspect, in the second temperature range, the carrier composition forms a gelled material comprising a network expanded throughout the carrier liquid.

In at least one aspect, the microcapsules are suspended in the carrier composition.

In at least one aspect, the carrier liquid includes an organic solvent.

In at least one aspect, the organic solvent includes a polar protic organic solvent.

In at least one aspect, the polar protic organic solvent includes a hydroxyl group.

In at least one aspect, the polar protic organic solvent is chosen from a list including polyethylene glycol, methanol, ethanol, propanol, butanol, pentadecanol, hexanol, decanol, undecanol, dodecanol, and tridecanol.

In at least one aspect, the gelling agent includes an amide.

In at least one aspect, the amide includes Dibutyl Ethylhexanoyl Glutamide, Dibutyl Lauroyl Glutamide, or combinations thereof.

In at least one aspect, the observable effect includes at least one effect selected from the group consisting of a color state change, a change in transparency, a change in hue, a change in an electrical property, a change in conductivity, a change in capacitance, a movement of the environmental indicator material along a media substrate, and combinations thereof.

In at least one aspect, the predetermined environmental condition includes at least one condition selected from the group consisting of temperature excursion above a predetermined temperature threshold for at least a predetermined amount of time, temperature excursion below a predetermined temperature for at least a predetermined amount of time, cumulative exposure to temperature over a time period above a predetermined threshold for at least a predetermined amount of time, exposure to a particular chemical, oxygen exposure, ammonia exposure, exposure to a particular chemical above a threshold concentration, exposure to a particular chemical above the threshold concentration for at least a predetermined amount of time, exposure to at least a predetermined amount of radiation of a particular type, ultraviolet light exposure, humidity exposure, exposure to a humidity level above a predetermined threshold, and exposure to a humidity level above a predetermined threshold for at least a predetermined amount of time.

In at least one aspect, at least a portion of the plurality of microcapsules are ruptured based on a force greater than a predetermined threshold, the force being at least one of a pressure force, a shear force, a frictional force, or a cutting force applied to the plurality of microcapsules.

In at least one aspect, the microcapsules include a diameter length between 20 to 250 μm.

In yet another general aspect, the present disclosure provides a rotary screen printing method including heating an ink composition to be within a first predetermined temperature range. The ink composition includes a first viscosity range at the first predetermined temperature range. The ink composition includes a carrier composition including a gelling agent and a carrier liquid. The ink composition further includes a plurality of microcapsules are suspended in the carrier composition. Each microcapsule encapsulating an environmental indicator material in a non-activated configuration and releasing the environmental indicator material when ruptured, the environmental indicator material configured, in response to exposure to a predetermined environmental condition, to change from an initial state to a second state producing an observable effect. The plurality of microcapsules prevent an occurrence of the observable effect while in the non-activated configuration and allow the occurrence of the observable effect when the environmental indicator material is exposed to the predetermined environmental condition after at least a portion of the plurality of microcapsules are ruptured. The method further includes inserting the heated ink composition into an internal volume of a rotating cylindrical screen, wherein the ink composition contacts an internal surface of the rotating cylindrical screen and is then brought into contact with a squeegee which acts to impel the heated ink composition through an array of apertures provided in the rotating cylindrical screen. The squeegee being fixedly disposed in the internal volume of the cylindrical screen so that the squeegee does not rotate within the cylindrical screen as the cylindrical screen is rotating. The method further includes contacting a printable substrate with an external surface of the rotating cylindrical screen to transfer a portion of the heated ink composition to the printable substrate through the apertures in the rotating cylindrical screen. The method further includes cooling the portion of the ink composition on the printable substrate to a second predetermined temperature range. The ink composition includes a second viscosity range at the second predetermined temperature range, and the second viscosity range is greater than the first viscosity range.

In at least one aspect, the method further includes mixing the gelling agent and the carrier liquid to form the carrier composition at a third predetermined temperature range. The third predetermined temperature range is greater than the first predetermined temperature range. In at least one aspect, the method further includes cooling the carrier composition to a temperature below the third predetermined temperature range and mixing the plurality of microcapsules into the carrier composition.

In at least one aspect, the ink composition comprises a third viscosity range at the third predetermined temperature range, the third viscosity range is less than the first viscosity range, and the third predetermined temperature range is greater than the first predetermined temperature range.

In at least one aspect, the third viscosity range is from 100 cps to 1000 cps and the third temperature range is greater than and equal to 100° C.

In at least one aspect, after the portion of the ink composition on the printable substrate is cooled, the carrier composition of the portion of the ink composition on the printable substrate forms a gelled material including a network expanded throughout the carrier liquid.

In at least one aspect, the carrier liquid includes an organic solvent.

In at least one aspect, the organic solvent includes a polar protic organic solvent.

In at least one aspect, the polar protic organic solvent includes a hydroxyl group.

In at least one aspect, the polar protic organic solvent is chosen from a list including polyethylene glycol, methanol, ethanol, propanol, butanol, pentadecanol, hexanol, decanol, undecanol, dodecanol, and tridecanol.

In at least one aspect, the gelling agent includes an amide.

In at least one aspect, the amide includes Dibutyl Ethylhexanoyl Glutamide, Dibutyl Lauroyl Glutamide, or combinations thereof.

In at least one aspect, a viscosity of the ink composition decreases as a temperature of the ink composition increases.

In at least one aspect, the first viscosity range is from 1000 cps to 10,000 cps and the second viscosity range is greater than 10,000 cps.

In at least one aspect, the first temperature range is from 50° C. to 80° C. and the second temperature range is less than 50° C.

In at least one aspect, the observable effect includes at least one effect selected from the group consisting of a color state change, a change in transparency, a change in hue, a change in an electrical property, a change in conductivity, a change in capacitance, a movement of the environmental indicator material along the printable substrate, and combinations thereof.

In at least one aspect, the predetermined environmental condition includes at least one condition selected from the group consisting of temperature excursion above a predetermined temperature threshold for at least a predetermined amount of time, temperature excursion below a predetermined temperature for at least a predetermined amount of time, cumulative exposure to temperature over a time period above a predetermined threshold for at least a predetermined amount of time, exposure to a particular chemical, oxygen exposure, ammonia exposure, exposure to a particular chemical above a threshold concentration, exposure to a particular chemical above the threshold concentration for at least a predetermined amount of time, exposure to at least a predetermined amount of radiation of a particular type, ultraviolet light exposure, humidity exposure, exposure to a humidity level above a predetermined threshold, and exposure to a humidity level above a predetermined threshold for at least a predetermined amount of time.

In at least one aspect, at least a portion of the plurality of microcapsules are ruptured based on a force greater than a predetermined threshold, the force being at least one of a pressure force, a shear force, a frictional force, or a cutting force applied to the printable substrate.

In at least one aspect, the microcapsules include a diameter length between 20 to 250 μm.

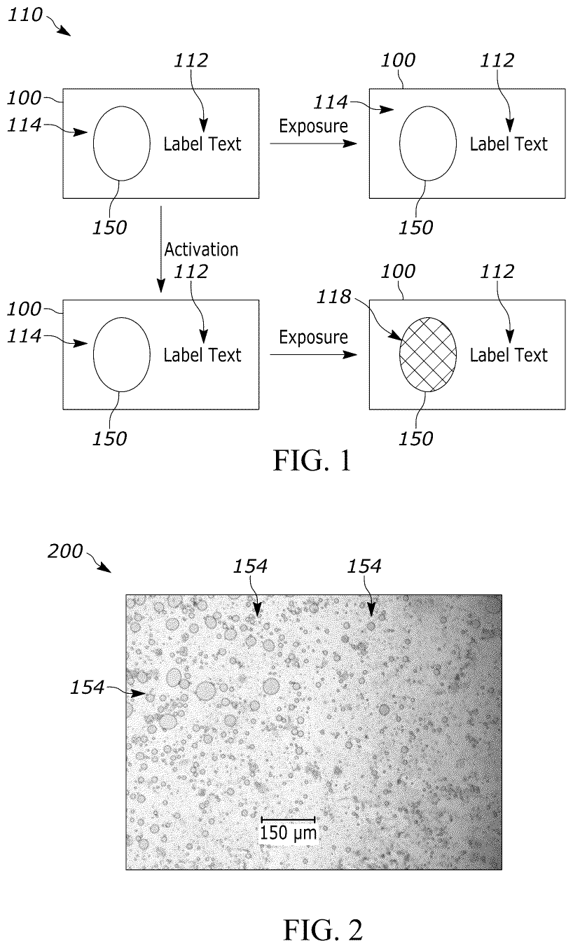

Referring mainly to FIGS. 1-5, microcapsules 154 encapsulating an indicator material 158 can be placed on a media element to form an activatable environmental sensor 150. The activatable environmental sensor 150 can be activated with physical forces such as pressure, sheer, cutting or friction sufficient to rupture the encapsulation and release the indicator material 158. The forces can be applied with a device, e.g. a media processing device, by a user pinching environmental sensor by hand, or any other means to apply a force to the media element. In some aspects, heat is also applied to the environmental sensor 150 for activation. In at least one aspect, the media can include a bursting additive (e.g. roughness and/or abrasiveness) to increase the ease of rupturing the indicator materials' encapsulation and activate the environmental sensor 150.

After the environmental indicator material 158 is exposed to a predetermined environmental condition, the environmental indicator material 158 can change from an initial material state to a second material state producing an observable effect. In at least one aspect, the change in material state from an initial material state to a second material state is a change in a property of the material, e.g. a state of matter, electrical conductivity, a color attributes, etc.

In at least one aspect, the microcapsules 154 prevent the observable effect from occurring in the environmental sensor 150 by protecting the environmental indicator material 158 from exposure to the predetermined environmental condition. For example, the environmental indicator material 158 can stay in the initial material state even if the microcapsules 154 are exposed to the predetermined environmental condition. However, after the microcapsules 154 are ruptured, the environmental indicator material 158 is released and any change in the material state of the environmental indicator material 158 upon exposure to the predetermined environmental condition can cause the observable effect to be produced by the environmental sensor. In an additional or alternative aspect, the microcapsules 154 prevent the observable effect from occurring in response to the predetermined environmental condition by containing the environmental indicator material 158 when it changes material state. For example, the environmental indicator material 158 can change material states within the microcapsule 154 but, while the material is contained in the microcapsules, the observable effect can be prevented, (e.g., by preventing movement of the material when it liquefies, or by hiding a color change of the material). After the microcapsules 154 are ruptured releasing the environmental indicator material 158, when the released environmental indicator material 158 is exposed to the predetermined environmental condition, the material may produce the observable effect that was prevented by containment in the microcapsules.

FIG. 1 illustrates a diagram 110 showing a media element 100 exposed to the predetermined environmental condition before and after activation, according to at least one aspect of the present disclosure. The media element 100 includes label text 112 and an environmental sensor 150. The environmental sensor 150 begins in a non-activated configuration, where the environmental sensor 150 is in an initial sensor state 114 before activation and exposure. After an activation process, the environmental sensor 150 is placed in the activated configuration, where the environmental indicator material 158 in the environmental sensor 150 has been released from at least a portion of the microcapsules 154. When the released environmental indicator material 158 is exposed to the predetermined environmental condition, then the environmental sensor 150 transitions from the initial sensor state 114 to a second sensor state 118. In at least one aspect, before activation, the environmental sensor 150 does not change sensor states even when exposed to the predetermined environmental condition.

The environmental indicator material 158 encapsulated in the microcapsules 154 can be different based on the desired environmental sensor 150. The environmental indicator material 158 can be a polymer, a polymer having side-chain crystallinity, an alkane wax, a polar protic material, a wax with a colorant, a gel with a colorant, an acid, a base, etc., or a combination thereof. Some example polar protic materials are polyethylene glycol, decanol, undecanol, dodecanol, and tridecanol. The type of environmental indicator material 158 used relates to the type of environmental sensor 150. Which allows the environmental indicator material 158 to be responsive to the appropriate predetermined environmental condition for the type of environmental sensor 150 desired. Some example predetermined environmental conditions are as follows: a temperature excursion above a predetermined temperature threshold for at least a predetermined amount of time, a temperature excursion below a predetermined temperature for at least a predetermined amount of time, a cumulative exposure to temperature over a time period above a predetermined threshold for at least a predetermined amount of time, an exposure to a particular chemical, an oxygen exposure, an ammonia exposure, an exposure to a particular chemical above a threshold concentration, an exposure to a particular chemical above the threshold concentration for at least a predetermined amount of time, an exposure to at least a predetermined amount of radiation of a particular type, an ultraviolet light exposure, a humidity exposure, an exposure to a humidity level above a predetermined threshold, an exposure to a humidity level above a predetermined threshold for at least a predetermined amount of time, and etc.

Once the environmental indicator material 158 is released from at least a portion of the microcapsules 154 (e.g., after an activation process ruptures the microcapsules 154) and is exposed to the predetermined environmental condition, then the released environmental indicator material 158 changes from an initial material state (e.g. corresponding to the initial sensor state 114) to a second material state (e.g. corresponding to the second sensor state 118) producing an observable effect. The observable effect may include a color state change, a change in transparency, a change in hue, a change in an electrical property, a change in conductivity, a change in capacitance, movement of the environmental indicator material 158 along a substrate, etc., or combinations thereof. In at least one aspect, the observable effect can be a continuous transition and, once a threshold in the continuous transition is exceeded, the environmental sensor 150 would be considered to go from the initial sensor state to the second sensor state.

The observable effect may be different based on the environmental indicator material 158 and the design of the media element 100. For example, an observable effect of movement of the environmental indicator material 158 along a substrate of the media element 100 can be caused by the substrate including a wick or microchannel that allows a liquid environmental indicator material 158 to move along the wick or microchannel (e.g., the environmental indicator material 158 can be liquid or have increased fluidity in the second material state). As another example, a change in an electrical property can be caused by an environmental indicator material 158 that melts above a specific temperature, which then allows the material to change an electrical property of a device, for example altering a dielectric in a capacitor changing capacitance, or, with a conductive liquid, completing, shorting, or breaking a circuit built into the substrate of the media element 100, which changes the electrical property.

The predetermined environmental condition required to generate the observable effect is based on the type of environmental sensor indicator 150. For example, with a temperature environmental sensor indicator the predetermined environmental condition can be a variety of different temperature ranges based on the specific temperature environmental sensor indicator. In at least one aspect, the predetermined environmental condition is between −5° C. and 5° C. For example, the predetermined environmental condition is preferably between −1° C. and 1° C. for thaw type environmental sensor indicators. In an alternative aspect, the predetermined environmental condition is between 5° C. and 15° C. For example, the predetermined environmental condition is preferably between 8° C. and 12° C. for some refrigeration type environmental sensor indicators. The design and materials of the environmental sensor indicator 150 can be chosen such that the predetermined environmental condition can be any temperature desired.

In at least one aspect, a diameter of each microcapsule 154 is between 20 μm to 250 μm. The shell thickness of each microcapsule 154 can range between 5 μm to 25 μm. FIG. 2 illustrates an example view 200 of a microcapsules 154 of the environmental sensor on the media element, according to at least one aspect of the present disclosure. The microcapsules 154 can be made of a variety of materials, e.g. urea formaldehyde, gelatin, protein, melamine, polyurea formaldehyde, gel, polymelamine formaldehyde, wax material, emulsions, etc. For example, polymelamine and polyurea formaldehyde are both used for encapsulations by interfavial polymerization which uses two immiscible phases. Once separated in the same vessel, a reaction is initiated at the interface of the two immiscible phases in the presence of an initiator and the material to be encapsulated. As polymerization occurs, microcapsules 154 form around the core material. The microcapsules 154 release the environmental indicator material 158 upon rupturing the microcapsules 154.

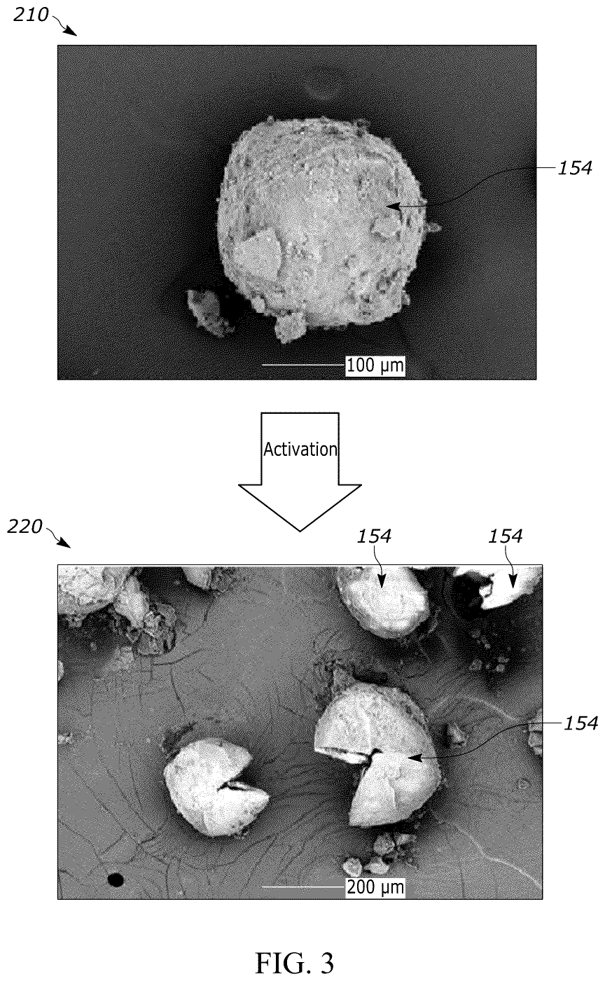

FIG. 3 illustrates a microcapsule 154 rupturing after an activation process, according to at least one aspect of the present disclosure. The view 210 illustrates a microcapsule 154 before activation and view 220 illustrates a ruptured microcapsule 154 after activation that released the environmental indicator material 158.

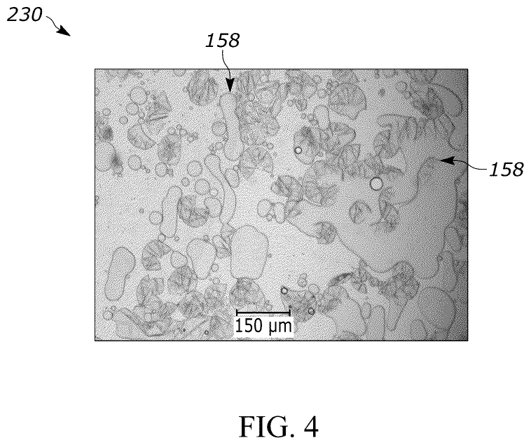

FIG. 4 illustrates an example view 230 of microcapsules 154 of the environmental sensor 150 on the media element 100, according to at least one aspect of the present disclosure. The environmental sensor 150 in FIG. 4 is in the activated configuration due to at least a portion of the microcapsules 154 having ruptured. As shown in FIG. 4 the environmental indicator material 158 has spread out and can experience the predetermined environmental condition outside of the microcapsules 154. In at least one aspect, the environmental indicator material 158 experiences the predetermined environmental condition and then the environmental indicator material 158 changes from an initial material state to a second material state producing an observable effect.

In at least one aspect, at least a portion of the microcapsules 154 are ruptured based on a force applied to the microcapsules 154, where the force exceeds or equals a predetermined threshold. The value of the predetermined threshold is based on the microcapsule material, thickness of the microcapsules 154, and/or the diameter of the microcapsules 154. The force can be a pressure force, a shear force, a frictional force, and/or a cutting force that cause at least a portion of the microcapsules 154 to rupture. In at least one aspect, the force is a pressure force and the predetermined threshold is between 1.5 pounds per square inch and 8 pounds per square inch. In an alternative aspect, the force is a pressure force and the predetermined threshold is between 4 pounds per square inch and 15 pounds per square inch. In yet another alternative aspect, the force is a pressure force and the predetermined threshold is between 16 pounds per square inch and 21 pounds per square inch. In yet another alternative aspect, the force is a pressure force and the predetermined threshold is based on the material properties and shell thickness of the microcapsules 154.

In at least one aspect, the portion of the microcapsules 154 are ruptured based, in part, on a temperature of the microcapsules 154, where the temperature of the microcapsules 154 exceeds or is equal to a predetermined temperature threshold. For example, temperature increases can cause the microcapsules 154 to rupture based on a minor amount of force being applied to the microcapsules 154. The value of the predetermined temperature threshold is based on the microcapsule material, the thickness of microcapsules 154, and/or the diameter of the microcapsules 154. In at least one aspect, the predetermined temperature threshold is between 90° C. and 110° C. In an alternative aspect, the predetermined temperature threshold is between 90° C. and 250° C.

In at least one aspect, at least a portion of the microcapsules 154 are ruptured based on both a force applied to the microcapsules 154 and a temperature of the microcapsules 154, where the force exceeds or equals a predetermined threshold and the temperature of the microcapsules 154 exceeds or is equal to a predetermined temperature threshold. The force can be a pressure force, a shear force, a frictional force, or a cutting force that cause at least a portion of the microcapsules 154 to rupture. In at least one aspect, the predetermined temperature threshold is between 90° C. and 110° C., the force is a pressure force, and the predetermined threshold is between 4 pounds per square inch and 15 pounds per square inch. In an alternative aspect, the predetermined threshold is between 1.5 pounds per square inch and 8 pounds per square inch.

In at least one aspect, each microcapsule 154 of the microcapsules 154 can have a first shell wall and a second shell wall encapsulating the environmental indicator material 158. For example, the first shell wall surrounds the environmental indicator material 158 and the second shell wall surrounds the first shell wall. Stated another way, there can be two coatings to each microcapsule 154 that surround the environmental indicator material 158. The first shell wall is configured to rupture based, in part, on a temperature being equal to or greater than a predetermined temperature threshold. The second shell wall is configured to rupture based on a force equal to or greater than a predetermined threshold being applied to the microcapsules 154. As discussed above, the force can be a pressure force, a shear force, a frictional force, or a cutting force that causes at least a portion of the microcapsules 154 to rupture. In this aspect, both heat and a force would need to be applied to the microcapsules 154 for at least a portion of microcapsules 154 to rupture.

The microcapsules 154 are configured to rupture in response to heat and/or force, and contain environmental indicator materials 158, e.g., the microcapsules 154 described in the commonly owned applications: U.S. patent application, titled “PRINTER ACTIVATABLE ENVIRONMENTAL SENSING THROUGH CHEMICAL ENCAPSULATION”, Attorney Docket No. 0820887.00357, application Ser. No. 18/369,498 filed Sep. 18, 2023; U.S. patent application, titled “MEDIA PROCESSING DEVICE AND COMPONENTS FOR ACTIVATABLE MEDIA PLATFORMS”, Attorney Docket No. 0820887.00358, application Ser. No. 18/369,520, filed Sep. 18, 2023; U.S. patent application, titled “USE OF ENCAPSULATED POLAR PROTIC CHEMISTRIES FOR RFID TEMPERATURE MONITORING”, Attorney Docket No. 0820887.00355, application Ser. No. 18/369,506, filed Sep. 18, 2023; U.S. patent application, titled “MEDIA CONSTRUCTION TO FACILITATE USE OF ACTIVATABLE PLATFORM”, Attorney Docket No. 0820887.00359, application Ser. No. 18/369,536, filed Sep. 18, 2023; and U.S. patent application, titled “RIBBON FOR USE IN PRODUCING PRINTER ACTIVATABLE INDICATORS”, Attorney Docket No. 0820887.00356, application Ser. No. 18/369,548, filed Sep. 18, 2023.

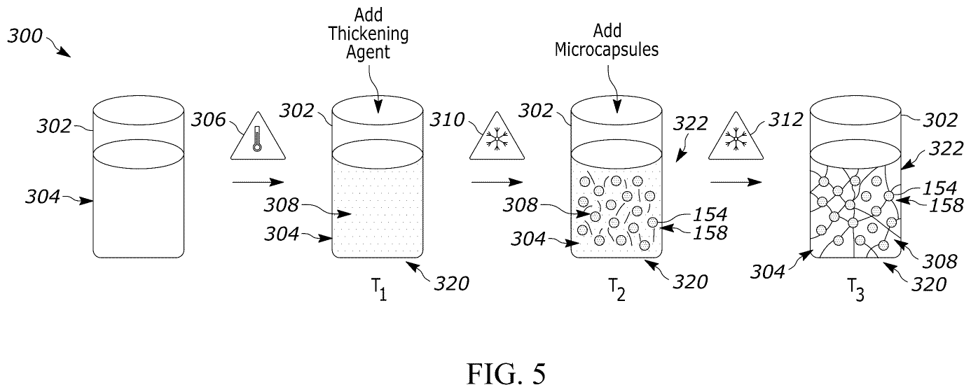

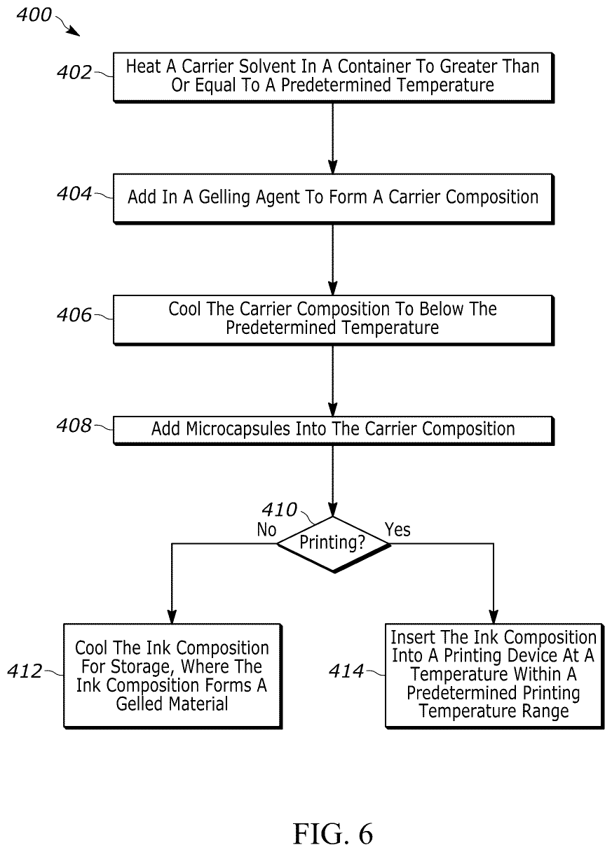

FIGS. 5 and 6 illustrate an example method 400 of making an ink composition 322 with a plurality of microcapsules 154 for screen printing, according to at least one aspect of the present disclosure. FIG. 5 is a schematic 300 of some of the steps of the method 400 shown in FIG. 6. The ink composition 322 includes a carrier composition 320 and a plurality of microcapsules 154. The carrier composition includes a carrier liquid 304 mixed with a gelling agent 308.

In at least one aspect, the carrier liquid 304 includes an organic solvent. In at least one aspect, the organic solvent includes a polar protic organic solvent. In at least one aspect, the polar protic organic solvent includes a hydroxyl group. Some non-limiting examples of a polar protic organic solvent are polyethylene glycol, methanol, ethanol, propanol, butanol, pentadecanol, hexanol, decanol, undecanol, dodecanol, and tridecanol.

In at least one aspect, the gelling agent 308 includes an amide, e.g. an amide gelling agent. Some example amides are Dibutyl Ethylhexanoyl Glutamide, Dibutyl Lauroyl Glutamide, or combinations thereof.

The method 400 provides an example method to create an ink composition 322 that includes a carrier composition 320 and microcapsules 154 encapsulating an indicator material 158. After the ink composition 322 is formed, the ink composition 322 can be used for printing (e.g. screen printing) an environmental sensor 150 on a media element (e.g. media element 100, FIG. 1).

The method 400 includes heating 402 a carrier liquid (e.g. carrier liquid 304) in a container to a temperature greater than or equal to a predetermined temperature. Referring to FIG. 3, a carrier liquid 304 is placed in a container 302. The carrier liquid 304 is heated 306 to be within a first temperature range T₁. In at least one aspect, the first temperature range T₁ has an upper bound of any temperature greater than 100° C. and a lower bound of 100° C. For example, the carrier liquid 304 can be heated to a temperature of 100° C. or above to be within the first temperature range.

The method 400 further includes adding 404 the gelling agent 308 while the carrier liquid 304 is heated to a temperature within the first temperature range T₁. In at least one aspect, the carrier liquid 304 penetrates into the gelling agent 308. For example, the gelling agent 308 can include fibers and the carrier liquid 304 penetrates/absorbs into the fibers of the gelling agent 308. The carrier liquid 304 mixed with the gelling agent 308 forms the carrier composition 320. At a temperature within the first temperature range T₁ (e.g. in at least one aspect, a temperature of 100° C. or greater), the carrier composition 320 has a first viscosity. In at least one aspect, the first viscosity is within a first viscosity range of 100 cps to 1000 cps.

The method 400 further includes cooling 406 the carrier composition 320 to below the predetermined temperature. The carrier composition 320 is cooled 310 to within a second predetermined temperature range T₂. As the carrier composition 320 cools, the gelling agent 308 begins to form an expanded network in the carrier liquid 304. For example, the carrier composition 320 begins to form associated networks within the carrier liquid 304 due to the gelling agent 308. In at least one aspect, the forming of the expanded network increases the viscosity of the carrier composition 320. As such, the viscosity of the carrier composition is dependent on the temperature of the carrier composition. For example, as the temperature of the carrier composition 320 increases the viscosity of the carrier composition 320 decreases and as the temperature of the carrier composition 320 decreases the viscosity of the carrier composition 320 increases. In at least one aspect, the second predetermined temperature range T₂ is 50° C. to 80° C. At a temperature within the second predetermined temperature range T₂, the carrier composition 320 has a second viscosity. In at least one aspect, the second viscosity is within a second viscosity range of 1000 cps to 10,000 cps.

The method further includes adding 408 a plurality of microcapsules 154 into the carrier composition 320. In at least one aspect, the microcapsules 154 are added to the carrier composition 320 when the carrier composition 320 is within the second predetermined temperature range T₂. In at least one aspect, the microcapsules 154 are suspended throughout the carrier composition 320 forming the ink composition 322. The ink composition 322 has a substantially similar viscosity to the carrier composition 320. For example, at a temperature within the second predetermined temperature range T₂, the viscosity of the ink composition 322 is within the second viscosity range of 1000 cps to 10,000 cps.

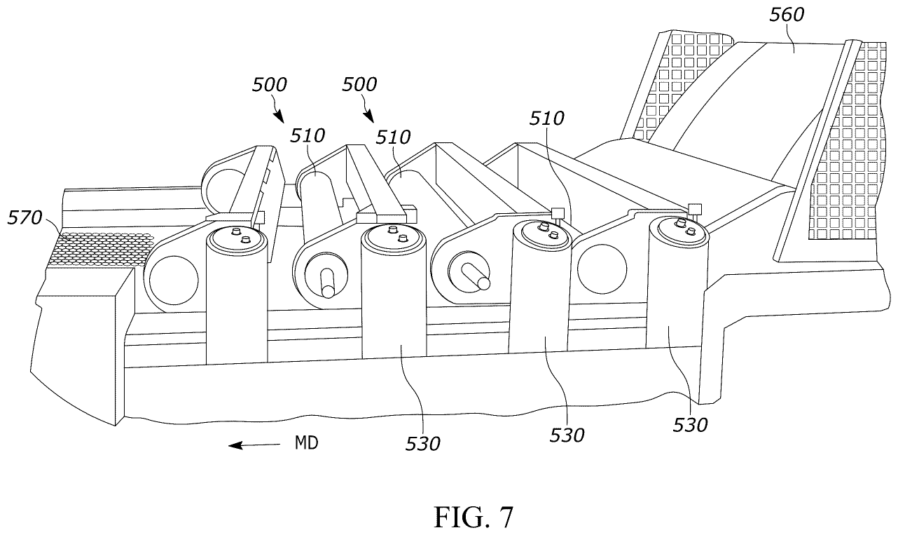

The method 400 further includes determining 410 if the ink composition is to be used for printing immediately, or stored for later use. If the ink composition is to be used for printing immediately, then the method 400 proceeds along the “yes” branch. The method further includes inserting 414 the ink composition 320 into a printing device at a temperature within a predetermined printing temperature range. In at least one aspect, the predetermined printing temperature range is the same as the second predetermined temperature range. In at least one aspect, the predetermined printing temperature range is 50° C. to 80° C. The ink composition 322 is printed at a temperature within the predetermined printing temperature range. FIGS. 7-13 discuss an example of screen printing with the ink composition 322.

Once the ink composition 322 is printed onto a media element (e.g. media element 100), then the ink composition 322 on the media element is allowed to cool. As the ink composition 322 cools 312, the expanded network formed from the gelling agent 308 in the carrier liquid 304 increases. For example, the associated networks within the carrier liquid 304 increase. As such, the viscosity of the ink composition 320 increases as the ink composition 322 cools. In at least one aspect, the ink composition 322 cools to within a third predetermined temperature range T₃. In at least one aspect, the third temperature range T₃ has an upper bound of 50° C. and a lower bound of any temperature below 50° C. For example, the ink composition 322 can be cooled to a temperature of 50° C. or below to be within the third predetermined temperature range T₃. At a temperature within the third predetermined temperature range T₃, the ink composition 322 has a third viscosity. In at least one aspect, the third viscosity is within a third viscosity range of greater than or equal to 10,000 cps. In at least one aspect, the ink composition 322 forms a gelled material at a temperature within the third predetermined temperature range T₃, where the gelling agent 308 is arranged as a network expanded throughout the carrier liquid 304. For example, the ink composition 322 forms associated networks within the carrier liquid 304 due to the gelling agent 308. In at least one aspect, the plurality of microcapsules 154 are suspended in the gelled material on the media element.

If the ink composition is to be printed later, then the method 400 proceeds along the “no” branch. The method 400 further includes cooling 412 the ink composition 322 for storage, where the ink composition 322 forms a gelled material. As discussed above, as the ink composition 322 cools 312, the expanded network formed from the gelling agent 308 in the carrier liquid 304 increases. As such, the viscosity of the ink composition 320 increases as the ink composition 322 cools. At a temperature within the third predetermined temperature range T₃, the ink composition 322 has a third viscosity. In at least one aspect, the third viscosity is within the third viscosity range of greater than or equal to 10,000 cps. In at least one aspect, the ink composition forms a gelled material at a temperature within the third predetermined temperature range T₃. As such, as the ink composition 322 cools for storage to a temperature within the third predetermined temperature range T₃, the ink composition 322 forms a gelled material. For example, the gelled material is made from the gelling agent 308 being arranged as a network expanded throughout the carrier liquid 304. Stated another way, the gelled material is formed due to associated networks within the carrier liquid 304 formed by the gelling agent 308. In at least one aspect, the plurality of microcapsules 154 are suspended in the gelled material.

When it is desired for the ink composition 322 to be printed, the ink composition 322 can be heated to within the predetermined printing temperature range (i.e. the second predetermined temperature range) to decrease the viscosity of the ink composition 322 for printing. In at least one aspect, as the ink composition 322 is heated the expanded network formed from the gelling agent 308 in the carrier liquid 304 decreases or breaks down allowing the viscosity of the ink composition 322 to decrease. Once the ink composition 322 is heated to within the predetermined printing temperature range, then the ink composition 322 can be inserted into a printing device. FIGS. 7-13 discuss an example of screen printing with the ink composition 322.

In at least one aspect, the concentration of the gelling agent 308 to the carrier liquid 304 in the carrier composition 320 is based on the specific gelling agent 308 and the specific carrier liquid 304 used. In some aspects, trial and error can be used to determine a concentration of the gelling agent 308 to the carrier liquid 304 that allows the carrier composition 320 to form a stable gelled material at a temperature within the third predetermined temperature range T₃. In at least one aspect, the carrier composition 320 is made of a concentration of at least 10% of the gelling agent 308 Dibutyl Ethylhexanoyl Glutamide mixed with the carrier liquid 304 dodecanol. In at least one alternative aspect, the carrier composition 320 is made of a concentration of at least 20% of the gelling agent 308 Dibutyl Lauroyl Glutamide mixed with the carrier liquid 304 dodecanol.

FIG. 7 is a perspective view of a rotary screen printing system comprising a plurality of rotary screen printing devices 500, according to at least one aspect of the present disclosure. FIG. 7 illustrates a rotary screen printing system that includes a plurality of rotary screen printing devices 500 arranged sequentially in a machine direction (MD). In at least one aspect, the rotary screen printing system includes a plurality of rotary screen printing devices 500, where one of the rotary screen printing devices 500 can be configured to print with the ink composition 322. In an alternative aspect, the rotary screen printing system has only one rotary screen printing device 500 and that one rotary screen printing device 500 can be configured to print with the ink composition 322.

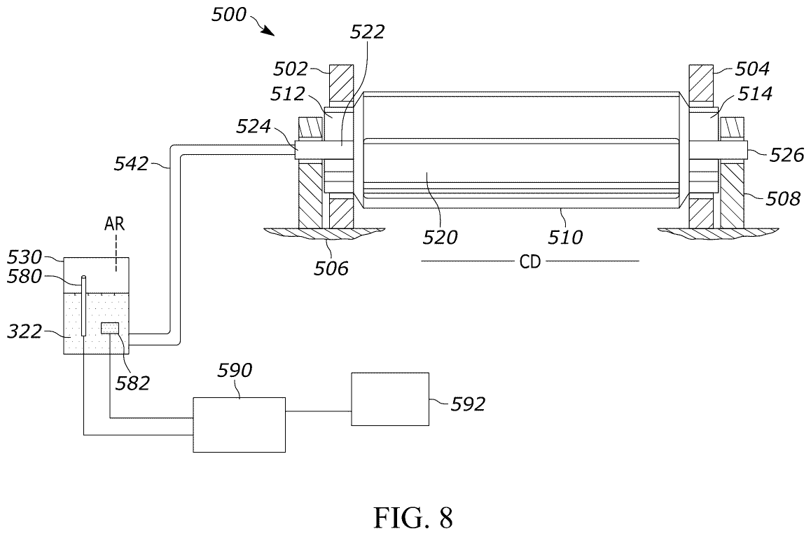

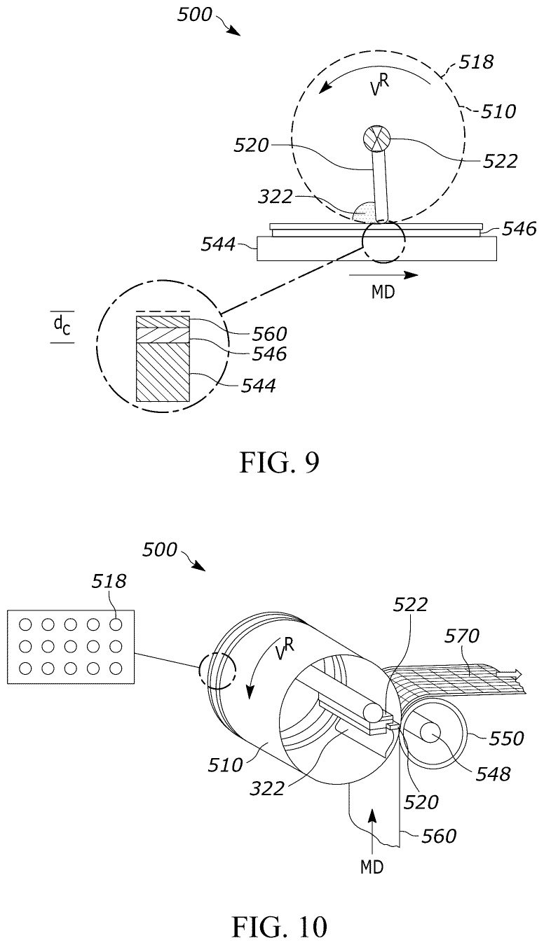

FIG. 8 is a cross-sectional view of a rotary screen printing device 500, according to at least one aspect of the present disclosure. FIG. 8 illustrates a cylindrical screen 510 of the rotary screen printing device 500. The cylindrical screen 510 is accommodated with ends 512, 514 respectively supported in a drive unit 502 and a support unit 504. The drive unit 502 is provided with a mechanism for rotatably driving the cylindrical screen 510. The support unit 504 may in some aspects incorporate a suspension or dampening device to minimize non-rotational movement of the cylinder screen 510 as it rotates at angular velocity V^{R} (FIGS. 9 and 10). A squeegee 520 extends across the cylindrical screen 510. The squeegee 520 is fixedly disposed within the internal volume of the cylindrical screen 510 such that it does not rotate with the cylindrical screen 510. The squeegee 520 is supported by a manifold 522 the ends 524, 526 that are accommodated respectively in supports 506, 508. A conduit 542 for the supply of the ink composition 322 from a reservoir 530 is connected to proximal end 524 of the manifold 522 which supports the squeegee 520. As discussed in regard to FIG. 5, the ink composition 322 includes a carrier composition 320 and a plurality of microcapsules 154.

In at least one aspect, one or more actuatable valves (not shown) may be disposed along the length of the conduit 542 to control the flow of the ink composition 322 between the reservoir 530 and the manifold 522. In at least one aspect, the flow is driven by gravity. In an alternative aspect, one or more pumps (not shown) may also be disposed along the length of the conduit 542 to drive the ink composition 322.

FIG. 9 is a sectional and detailed view of the rotary screen printing device 500, according to at least one aspect of the present disclosure. FIG. 9 provides a sectional view, parallel to the machine direction (MD) through the cylindrical screen 510 of a rotary screen printing device 500. A detailed view of the configuration of the substrate 560 to be printed is also provided therein. In at least one aspect, the substrate 560 includes a media element (e.g. media element 100).

As shown, the squeegee 520 is disposed within the cylindrical screen 510 by attachment to a manifold 522: the squeegee 520 and its associated manifold 522 both extend across the width of the cylindrical screen 510. The manifold 522 is disposed at or beneath the central axis of the rotary cylindrical screen 510 and is provided with at least one central cavity which receives the ink composition 322 from the conduit 542 to which it is operatively connected. The manifold 522 is further provided with one or more openings (not shown) which extend through the manifold's external surface to the cavity thereof. The ink composition 322 may pass through said openings and is thereby delivered to an internal surface of the rotary cylindrical screen 510.

The illustrated shape of the squeegee 520 in FIG. 9 is not intended to be limiting: rather the squeegee may possess any operative shape that is capable of spreading the ink composition 322 on the internal surface of the rotary cylindrical screen 510. The squeegee 520 will conventionally possess sufficient flexibility such that, when its leading edge is disposed to contact a moving surface, the squeegee traverses that surface. Exemplary materials from which the squeegee may be constructed include: alloys, such as spring steel or stainless steel; fluoropolymers such as a Teflon® polytetrafluoroethylene based composition; and, elastomers such as a silicone elastomers.

The squeegee 520 may be urged into contact with the internal surface of the rotary cylindrical screen 510 by a source of pressure (not shown), which source may, for example, be a mechanical, electromechanical, pneumatic or magnetic source. It is envisaged that the pressure exerted on the squeegee 520 may be adjusted based on inter alia the resiliency of the squeegee, the amount of ink composition 322 to be applied and the printing definition which is required. Under the action of the squeegee 520, the ink composition 322 is extruded from rotary cylindrical screen at a temperature T_{A}. In at least one aspect, the temperature T_{A} is within the predetermined printing temperature range (i.e. the second predetermined temperature range), as discussed in regard to FIGS. 5 and 6. In at least one aspect, the predetermined printing temperature range is 50° C. to 80° C.

The cylindrical screen 510 is provided with a multiplicity of apertures 518—also shown in FIG. 10—which extend substantially across the cylindrical screen and which, in combination, are determinative of the pattern 570 (FIGS. 7 and 10) of ink composition which is applied to the substrate 560 using that specific screen 510. In a printing system comprising more than one such rotary cylindrical screen 510, such as that illustrated in FIG. 7, the configuration of the apertures 518 for each screen 510 will be adapted to achieve a particular final ink pattern 570 upon synchronization of the rotational speeds (V^{R}) of the screens, the relative positions of the rotary screens and the velocity of the substrate 560 in the machine direction (MD). That aside, there is no particular intention to limit the configuration of the apertures 518 in the rotary cylindrical screen 510 and thereby the ink pattern obtained.

In at least one aspect, the apertures 518 can be positioned to place an environmental sensor 150 on a media substrate 560. The media substrate 560 can include a plurality of media elements 100. The apertures 518 can be positioned such that one or more environmental sensors are positioned on each media element 100 of the media substrate 560.

In a printing system that includes a plurality of rotary screens, the configuration of the apertures 518 of each screen may be independently determined. However, in some aspects, rotary screens disposed sequentially in the machine direction may be provided with the same configuration of apertures: this may enable the ink composition 322 to be applied by each rotary screen to be deposited in the same pattern and, optionally, to be deposited in a laminar manner by each sequential rotary screen.

Independently of the configuration of the apertures 518, the rotary cylindrical screen 510 may in some embodiments be characterized by at least one of: an effective open area of from 1% to 50% in one aspect, from 5% to 50% in an alternative aspect, or from 5% to 30% in yet another alternative aspect; an effective aperture area of from 0.1 mm² to 15 mm² in one aspect or from 0.5 mm² to 10 mm² in an alternative aspect; an aperture aspect ratio (e.g. defined as the ratio of the major axis to the minor axis of a single aperture) from 1 to 15 in one aspect, from 1 to 10 in an alternative aspect, or from 1 to 5 in yet another alternative aspect; and an aperture density of at least 150 in one aspect or at least 300 in an alternative aspect. In at least one aspect, these characterizations are not mutually exclusive: one, two, three or four of these characterizations may be applied.

It will be appreciated that a variety of approaches may be employed to provide the substrate 560 to the rotary screen printing apparatus. For example, the provision of the substrate 560 may be: continuous, such as being provided from a roll stock or a stack; semi-continuous; on-demand; or, discontinuous, such as being provided in discrete units.

The provided substrate 560 moves in the machine direction (MD) at a speed such that it passes under the rotary cylindrical screen 510 and receives an effective amount of the ink composition 322 without substantial deviations. Deviation is meant as any undesired attribute within the print including but not limited to variant thickness of the ink composition 322, line breaks, smudges, and other ostensible departures from the pre-determined pattern 570 (FIGS. 7 and 10). Illustrative speeds of the substrate in the machine direction (MD) are from 0.1 ms⁻¹ to 1 ms⁻¹ or in some alternative aspects 0.1 ms⁻¹ to 0.5 ms⁻¹.

The rotary cylindrical screen 510 of FIG. 9 is depicted as contacting the substrate 560 which is carried in the machine direction (MD) by a horizontal planar shuttle 544. A laminar support 546—sometimes referred to in the art as a blanket or wafer—is interposed between the shuttle 544 and the substrate 560. It will be acknowledged that such a laminar support 546 is optional. The gap d_{c} between the external surface of the rotary cylindrical screen 510 and the upper surface of the shuttle 544, and the surface tension and resiliency of the substrate 560, shuttle 544, and laminar support (546, if present) are each partially determinative of the pressure at the point of contact of the rotary cylindrical screen 510 and the substrate 560 and thereby also determinative of the volume of the ink composition 322 transferred to the substrate 560.

Alternative configurations of shuttle 544 and rotary cylindrical screen 510 will be known to those of ordinary skill in art. The shuttle 544 may, for instance, convey the substrate 560 between the rotary cylindrical screen 510 and a counter roller: the movement of the shuttle 544 and the carried substrate need not be in the horizontal plane, as illustrated in for example U.S. Pat. No. 6,098,546A. FIG. 10 is a sectional view of an alternative rotary screen printing configuration compared to the rotary screen printing device 500, according to at least one aspect of the present disclosure. FIG. 10 provides an alternative configuration wherein the substrate 560 is passed between the rotary cylindrical screen 510 and an impression roller 550. A plate cylinder drive 548 is disposed within the impression roller 550.

As described herein above, the rotary screen printing device 500 may in some aspects include at least one heater (not shown) of which the output heats the rotary screen cylinder 510. The form of this heater is not intended to be limited and may include: a heated gas stream provided to the internal volume of the rotary cylindrical screen 510; a radiant heater disposed within the internal volume of the rotary cylindrical screen 510; a radiant heater disposed external to the rotary cylindrical screen 510; an induction heater disposed external to the rotary cylindrical screen 510; or, an incubative heater which surrounds or encloses at least a portion of the rotary cylindrical screen 510. Exemplary heaters for generating heat energy for direct absorption by the cylindrical screen 510 are described in: U.S. Pat. No. 4,693,179 (Watts et al.); WO85/03672 (Lockwood Technical, Inc.); US 2011/0079156 (Bartesaghi); Netherlands Patent No. 10 30 670 C (Stork Prints B.V. te Boxmeer); and, U.S. Pat. No. 5,772,763 B1 (Hines, Jr. et al.).

In at least one aspect, the heater is used to heat the ink composition 322 in the rotary cylindrical screen 510 to within the predetermined printing temperature range (i.e. the second predetermined temperature range), as discussed in regard to FIGS. 5 and 6. In at least one aspect, the predetermined printing temperature range is 50° C. to 80° C. As discussed previously, the ink composition 322 at a temperature within the predetermined printing temperature range has a viscosity in the second viscosity range. In at least one aspect, the second viscosity range is 1000 cps to 10,000 cps.

As depicted in FIGS. 7 and 8, a reservoir 530 is provided for the ink composition 322: a conduit 542 provides fluid communication between the rotary screen cylinder 510 and the reservoir 530. While only one such reservoir is illustrated per screen 510, it will be appreciated that more than one reservoir 530 may actually be coupled to a given rotary cylindrical screen 510.

As used herein, the term “reservoir” includes a receptacle, chamber or similar vessel for containing an ink composition for supply to the rotary screen printing device 500. The reservoir 530 of the rotary screen printing device 500 may be equipped with an agitator which herein refers to a mechanism that imparts motion to the contents of the reservoir 530. That motion may be sufficient to ensure that any solid components (e.g. microcapsules 154) within the ink composition are placed into or maintained in suspension prior to the supply of the ink composition 322 to the rotary screen printing device 500. The agitation may, in certain aspects, be sufficient to ensure the ink composition is homogeneous. As is known in the art, agitation may be provided continuously, periodically or as a single action: the energy and, if applicable, the frequency of agitation may be moderated for a given ink composition disposed in the reservoir 530.

In at least one aspect, the agitator includes at least one paddle element configured to rotate about an axis parallel to the longitudinal (vertical) axis A_{R} of the reservoir and the at least one paddle element is configured to extend out of a primary plane of the agitator. In this configuration, the rotation of the paddle element will generally provide circumferential and/or radial mixing of the ink composition 322 in a substantially horizontal direction. The efficacy of mixing throughout the ink composition 322 contained in the reservoir 530 may be improved by providing a plurality of said paddle elements disposed vertically in a direction generally parallel to the rotational axis of the agitator. The cross-sectional profile of the paddle element may vary for each paddle element. The cross-sectional profile may, for example, be independently selected from the group consisting of: elliptical; annular; and, quadrilateral.

Referring again to FIG. 8, the rotary screen printing device 500 includes a reservoir 530 to hold the ink composition 322 and a heater 582 is provided which heats the ink composition 322 held therein to within a predetermined printing temperature range. The ink composition 322 at a temperature within the predetermined printing temperature range has a viscosity that allows the ink composition 322 to be screen printed. In at least one aspect, the viscosity is within a viscosity range defined as 1000 cps to 10,000 cps. In at least one aspect, the predetermined printing temperature range is 50° C. to 80° C. In at least one aspect, a temperature sensor 580 to measure a temperature of the ink composition 322 is coupled to the reservoir 530. It will be appreciated that the form and positions of the heater 582 and temperature sensor 580 shown in FIG. 8 are illustrative only.

The conduit 542 provides fluid communication of the ink composition 322 between the reservoir 530 and the internal volume of the rotatable cylindrical screen 510 having, as described above, an internal volume and an array of apertures 518 there through. A squeegee 520 is fixedly disposed in the internal volume of the cylindrical screen 510 so that the squeegee 520 does not rotate within the cylindrical screen 510 when the cylindrical screen 510 is rotated.

In at least one aspect, the conduit 542 is further provided with a second heater (not shown) to heat the conduit 542 and a second heat sensor (not shown) to detect one of the temperature of the conduit 542, the temperature of the ink composition 322 passing through the conduit 542, or the temperature of the ink composition 322 discharged from the conduit 542 to the internal volume of the rotary cylindrical screen 510. Such a heating system is described in commonly owned U.S. patent application, titled “ROTARY SCREEN PRINTING APPARATUS AND PROCESS”, Attorney Docket No. 0820887.00337, application Ser. No. 17/947,927 filed Sep. 19, 2022.

A controller 590, optionally connected to a power source 592, is in communication with the temperature sensor 580 and the heater 582. Via the controller 590, the energy provided by the heater 582 may thus be controlled to attain or maintain specific temperature conditions of the ink composition 322 held in the reservoir 530. In particular, the controller 590, receiving data from the temperature sensor 580, instructs the heater 582 to expend sufficient energy so that the temperature of the ink composition 322 held in the reservoir is maintained within the predetermined printing temperature range where the ink composition 322 has a viscosity within the second viscosity range.

The temperature sensor 580 may be disposed within the ink composition 322 respectively held within the reservoir 530. The temperature sensor 580 for this aspect may include thermometers, such as dip stick thermometers. Alternatively, the temperature sensor 580 may be disposed upon an inner surface of the reservoir 530 at a location such that ink composition 322 is within the temperature sensing zone of the sensor. For example, an infrared sensor may be disposed on an internal surface of the reservoir 530 facing but not being immersed in the ink composition 322. It is further considered that the temperature sensor 580 may, in some aspects, be disposed on an external surface of the reservoir 530.

The form of the heater 582 is not intended to be limited. As such, it is envisaged that the heater 582 may be one or more of: an incubative heater; a radiative heater directed at the reservoir contents; an immersed heater; or, an inductive heater.

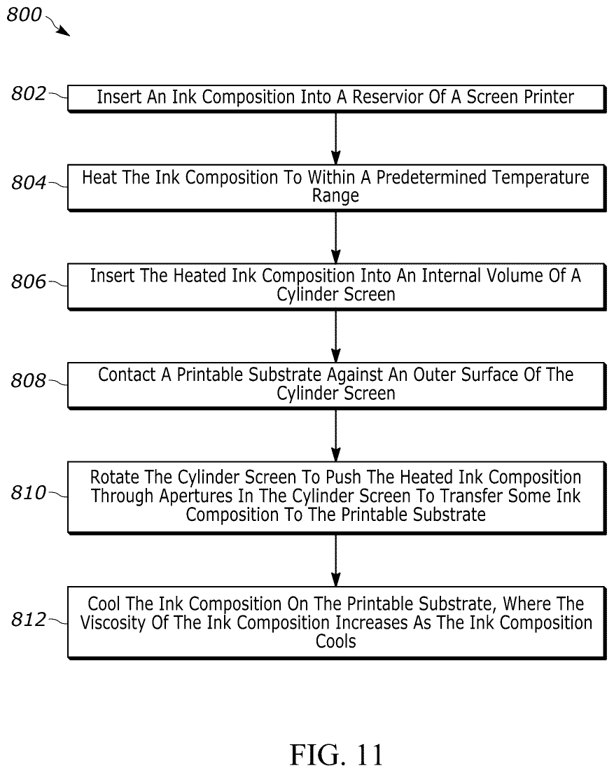

FIG. 11 illustrates an example method 800 for screen printing an ink composition 322 with a plurality of microcapsules 154, according to at least one aspect of the present disclosure. The method 800 includes inserting 802 the ink composition 322 into the reservoir 530 of the screen printing device 500. The method 800 further includes heating 804 the ink composition 322 to within a predetermined printing temperature range. In at least one aspect, the predetermined printing temperature range is defined as 50° C. to 80° C. The ink composition 322 at a temperature within the predetermined printing temperature range has a viscosity that allows the ink composition 322 to be screen printed. In at least one aspect, the viscosity is within a viscosity range defined as 1000 cps to 10,000 cps.

The method 800 further includes inserting 806 the heated ink composition 322 into the internal volume of the cylinder screen 510. The method 800 further includes contacting 808 a printable substrate 560 against an outer surface of the cylinder screen 510. The method 800 further includes rotating 810 the cylinder screen to push the heated ink composition 322 through apertures 518 in the cylinder screen 510 to transfer some ink composition 322 to the printable substrate 560.

The method 800 further includes cooling 812 the ink composition 322 on the printable substrate 560 where the viscosity of the ink composition 322 on the printable substrate 560 increases as the ink composition 322 cools. In at least one aspect, the ink composition 322 on the cools to a temperature within the third temperature range T₃ (FIG. 5). In at least one aspect, the third temperature range T₃ has an upper bound of 50° C. and a lower bound of any temperature below 50° C. For example, the ink composition 322 can be cooled to a temperature of 50° C. or below to be within the third predetermined temperature range T₃. At a temperature within the third predetermined temperature range T₃, the ink composition 322 has a viscosity within a viscosity range defined as greater than or equal to 10,000 cps. In at least one aspect, the ink composition 322 forms a gelled material at a temperature within the third predetermined temperature range T₃. For example, the gelled material is made from the gelling agent 308 being arranged as a network expanded throughout the carrier liquid 304. In at least one aspect, the plurality of microcapsules 154 are suspended in the ink composition 322 on the media element. For example, the plurality of microcapsules 154 can be suspended in the gelled material on the media element.

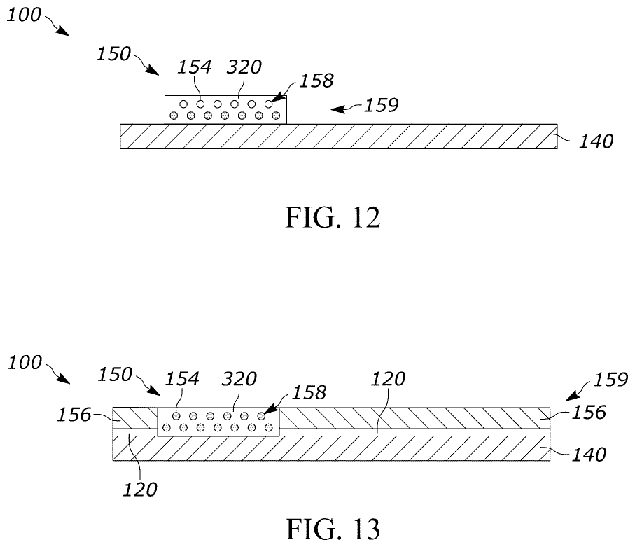

FIG. 12 illustrates a diagram of a cross-section of an example media element including an environmental sensor and FIG. 13 illustrates the diagram of FIG. 12 with filler material, both according to at least one aspect of the present disclosure. Media element 100 is one non-limiting example of a media element. The media element 100 is an activatable media element. Some examples of media elements 100 that can include the microcapsules 154 encapsulating indicator material 158 can include, for example, labels, tags, wristbands, inlays, plastic cards, etc.

In at least one aspect, the environmental sensor 150 can be printed onto the media element 100 through screen printing the ink composition 322 onto the media substrate 140 (or media substrate 560). For example, the method 800 (FIG. 11) can be used to print the ink composition 322 onto the media substrate 140. The environmental sensor 150 is made of the ink composition 322 attached to the media element 100. As discussed in regard to FIG. 5, the ink composition 322 includes a carrier composition 320 and the plurality of microcapsules 154 that are suspended in the carrier composition 320. In at least one aspect, when the environmental sensor 150 is at a temperature at or below 50° C., the carrier composition 320 forms into a gelled material. The gelled material is made from the gelling agent 308 being arranged as a network expanded throughout the carrier liquid 304. The plurality of microcapsules 154 are suspended in the gelled material on the media element 100. In at least one aspect, the gelled material has a viscosity greater than or equal to 10,000 cps.

In at least one aspect, the media element 100 is made of layers attached to each other. In some aspects, all or part of each layer is attached with an adhesive 120 (FIG. 13). In at least one aspect, one of the layers is a paper or polymer material (e.g. media substrate 140). One of the layers is the environmental sensor layer 159 that includes microcapsules 154 encapsulating an indicator material 158 to form the environmental sensor 150 on the media element 100. The environmental sensor 150 is adhered to the media substrate 140 due to the functionality possessed by the carrier composition 320 of the ink composition 322. For example, different functionality provides better wetting and anchorage of the carrier composition 320 to the media substrate 140. In at least one aspect, the carrier composition 320 of the ink composition 322 has amide functionality that assists with adhesion to the media substrate 140. For example, the amide functionality can adhere the ink composition 322 to the media substrate 140.

In at least one aspect, the media substrate 140 material is a paper material, polyester material, thermoplastic and/or vinyl polymer materials, such as polypropylene, polyethylene, polyethylene terephthalate, nylon, and/or Tyvek®, and/or any combination thereof. Some examples of media elements 100 that can include the encapsulated indicator material 158 are labels, tags, wristbands, inlays, plastic cards, and etc.

The releasing of the environmental indicator material 158 transitions the environmental sensor 150 on the media element 100 from a non-activated configuration to an activated configuration. Once in the activated configuration, the environmental sensor 150 can change from an initial sensor state to a second sensor state producing an observable effect upon exposure of the environmental indicator material 158 to the predetermined environmental condition as discussed in regard to FIGS. 2-4.

In at least one aspect, the carrier composition 320 allows the environmental indicator material 158 to flow through the carrier composition 320 when the environmental indicator material 158 is released from the microcapsules 154. For example, some environmental sensors 150 include a wicking material to move the indicator material 158 along the media substrate 140 and carrier composition 320 allows the environmental indicator material 158 to flow to the wicking material. In at least one aspect, the carrier composition 320 does not interact with the environmental indicator material 158.

Referring to FIG. 13, in some aspects, the environmental sensor layer 159 further includes a filler material 156 (e.g., to provide a generally uniform thickness of the media element 100). In some aspects, the filler material 156 can be part of the environmental sensor 150 (e.g. a wicking material to move the indicator material 158 along the media substrate 140). In an additional/alternative aspect, the filler material 156 is used to bridge the gap between layers formed by the thickness of the microcapsules 154 (e.g. the filler material can be a paper or a polymer material) so that a thickness of the media element 100 is uniform. In at least one aspect, the filler material 156 is based on the type of environmental sensor 150 attached to the media element 100. The filler material 156 can include an adhesive layer 120 that allows the filler material 156 to be attached to the media substrate 140.

In some aspects, the environmental sensor layer 159 may have a lamination layer (not shown) placed over the environmental sensor layer 159. The lamination layer may be added if the environmental sensor layer 159 includes filler material 156 or if it does not include filler material 156. In some aspects, the lamination layer can be used to hold the environmental sensor 150 and/or released indicator material 158 in position.

In various aspects, the microcapsules 154 in the ink composition 322 can be replaced with solid particles to form a new ink composition. In at least one aspect, the solid particles are conductive particles (e.g. copper, silver, etc.). The new ink composition includes the carrier composition 320 and solid particles suspended in the carrier composition 320. In at least one aspect, the viscosity properties of the new ink composition are substantially similar to the viscosity properties of the ink composition 322.

All the methods previously discussed (e.g, methods 400 and 800) would function the same with the new ink composition. Similarly, the new ink composition can also be printed the same as the ink composition 322. For example, the screen printing device 500 can be used to print the new ink composition. Additionally, since the new ink composition includes the same carrier composition 320, the viscosities of the new ink composition at the temperature ranges T₁, T₂, and T₃ are the same as discussed in regard to FIG. 5. Similar to the ink composition 322, in at least one aspect, the new ink composition can be printed on a media element through screen printing. As the new ink composition cools to below 50° C., the new ink composition forms a gelled material on the media element where the gelling agent (e.g. gelling agent 308) is arranged as a network expanded throughout the carrier liquid (e.g. carrier liquid 304). In at least one aspect, the solid particles are suspended in the gelled material that is adhered to a media element.

In at least one aspect, the solid particles in the new ink composition are conductive particles. In some aspects, the new ink composition can be printed onto a media element as part of an environmental sensor on the media element. In at least one aspect, the environmental sensor is a temperature environmental sensor. In this aspect, the media element includes an electrical component and the new ink composition is printed onto at least part of the electrical component. In some aspects, a non-conductive coating can be placed on the electrical component before printing the new ink composition. As the new ink composition cools to below 50° C., the new ink composition forms a gelled material on the media element and the solid particles are suspended in the gelled material. As the temperature rises, the viscosity of the gelled material decreases and once a predetermined temperature threshold is reached the solid particles are able to pass through the new ink composition to contact the electrical component on the media element. In at least one aspect, a non-conductive coating on the electrical component liquefies above the predetermined temperature and allows the solid particles to pass through the non-conductive coating to contact the electrical component. The solid particles contacting the electrical component can change an electrical property of the electrical component indicating that the temperature has increased above the predetermined threshold. A temperature environmental sensor is one non-limiting example of an environmental sensor that can be created by printing the new ink composition.

In the foregoing detailed description, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

The foregoing detailed description has set forth various forms of the systems and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

Any of the software components or functions described in this application, may be implemented as software code to be executed by a processor using any suitable computer language such as, for example, Python, Java, C++ or Perl using, for example, conventional or object-oriented techniques. The software code may be stored as a series of instructions, or commands on a computer readable medium, such as RAM, ROM, a magnetic medium such as a hard-drive or a floppy disk, or an optical medium such as a CD-ROM. Any such computer readable medium may reside on or within a single computational apparatus, and may be present on or within different computational apparatuses within a system or network.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

Unless specifically stated otherwise as apparent from the foregoing disclosure, it is appreciated that, throughout the present disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the terms “component,” “system,” “module” and the like can refer to a computer-related entity, either hardware, a combination of hardware and software, software, or software in execution.

It will be appreciated that some embodiments may be comprised of one or more specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions can be implemented by a state machine that has no stored program instructions, or in one or more application specific integrated circuits (ASICs), in which each function or some combinations of certain of the functions are implemented as custom logic. Of course, a combination of the two approaches can be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A media element, comprising:
a media substrate;
a gelled material adhered to the media substrate; and
a plurality of microcapsules suspended in the gelled material, each microcapsule encapsulating an environmental indicator material in a non-activated configuration and releasing the environmental indicator material when ruptured, the environmental indicator material configured, in response to exposure to a predetermined environmental condition, to change from an initial state to a second state producing an observable effect, wherein the plurality of microcapsules prevent an occurrence of the observable effect while in the non-activated configuration, and allow the occurrence of the observable effect when the environmental indicator material is exposed to the predetermined environmental condition after at least a portion of the plurality of microcapsules are ruptured.

2. The media element of claim 1, wherein the gelled material comprises a network expanded throughout a carrier liquid.

3-6. (canceled)

7. The media element of claim 2, wherein the gelled material comprises a gelling agent comprising an amide.

8. (canceled)

9. The media element of claim 1, wherein the gelled material comprises a viscosity of greater than or equal to 10,000 cps at a temperature below 50° C.

10. (canceled)

11. The media element of claim 1, wherein the environmental indicator material flows through the gelled material when released.

12. The media element of claim 1, wherein the observable effect comprises at least one effect selected from the group consisting of a color state change, a change in transparency, a change in hue, a change in an electrical property, a change in conductivity, a change in capacitance, a movement of the environmental indicator material along the media substrate, and combinations thereof.

13. The media element of claim 1, wherein the predetermined environmental condition comprises at least one condition selected from the group consisting of temperature excursion above a predetermined temperature threshold for at least a predetermined amount of time, temperature excursion below a predetermined temperature for at least a predetermined amount of time, cumulative exposure to temperature over a time period above a predetermined threshold for at least a predetermined amount of time, exposure to a particular chemical, oxygen exposure, ammonia exposure, exposure to a particular chemical above a threshold concentration, exposure to a particular chemical above the threshold concentration for at least a predetermined amount of time, exposure to at least a predetermined amount of radiation of a particular type, ultraviolet light exposure, humidity exposure, exposure to a humidity level above a predetermined threshold, and exposure to a humidity level above a predetermined threshold for at least a predetermined amount of time.

14-15. (canceled)

16. An ink composition for screen printing, the ink composition comprising:
a carrier composition comprising:
a gelling agent; and
a carrier liquid; and
a plurality of microcapsules, each microcapsule encapsulating an environmental indicator material in a non-activated configuration and releasing the environmental indicator material when ruptured, the environmental indicator material configured, in response to exposure to a predetermined environmental condition, to change from an initial state to a second state producing an observable effect, wherein the plurality of microcapsules prevent an occurrence of the observable effect while in the non-activated configuration, and allow the occurrence of the observable effect when the environmental indicator material is exposed to the predetermined environmental condition after at least a portion of the plurality of microcapsules are ruptured.

17-18. (canceled)

19. The ink composition of claim 16, wherein a viscosity of the carrier composition decreases as a temperature of the carrier composition increases.

20. The ink composition of claim 19, wherein the carrier composition comprises a first viscosity range associated with a first temperature range and a second viscosity range associated with a second temperature range, wherein values in the first temperature range are greater than values in the second temperature range, and wherein the second viscosity range is lower than the first viscosity range.

21. The ink composition of claim 20, wherein the first temperature range is from 50° C. to 80° C. and the second temperature range is less than 50° C., and wherein the first viscosity range is from 1000 cps to 10,000 cps and the second viscosity range is greater than 10,000 cps.

22. (canceled)

23. The ink composition of claim 20, wherein in the second temperature range the carrier composition forms a gelled material comprises a network expanded throughout the carrier liquid.

24. (canceled)

25. The ink composition of claim 16, wherein the carrier liquid comprises an organic solvent.

26. The ink composition of claim 25, wherein the organic solvent comprises a polar protic organic solvent.

27. (canceled)

28. The ink composition of claim 26, wherein the polar protic organic solvent is chosen from a list comprising polyethylene glycol, methanol, ethanol, propanol, butanol, pentadecanol, hexanol, decanol, undecanol, dodecanol, and tridecanol.

29. The ink composition of claim 16, wherein the gelling agent comprises an amide.

30. The ink composition of claim 29, wherein the amide comprises Dibutyl Ethylhexanoyl Glutamide, Dibutyl Lauroyl Glutamide, or combinations thereof.

31-34. (canceled)

35. A rotary screen printing method, comprising:
heating an ink composition to be within a first predetermined temperature range, wherein the ink composition comprises a first viscosity range at the first predetermined temperature range, and wherein the ink composition comprises:
a carrier composition comprising:
a gelling agent; and
a carrier liquid; and
a plurality of microcapsules are suspended in the carrier composition, each microcapsule encapsulating an environmental indicator material in a non-activated configuration and releasing the environmental indicator material when ruptured, the environmental indicator material configured, in response to exposure to a predetermined environmental condition, to change from an initial state to a second state producing an observable effect, wherein the plurality of microcapsules prevent an occurrence of the observable effect while in the non-activated configuration, and allow the occurrence of the observable effect when the environmental indicator material is exposed to the predetermined environmental condition after at least a portion of the plurality of microcapsules are ruptured;
inserting the heated ink composition into an internal volume of a rotating cylindrical screen, wherein the ink composition contacts an internal surface of the rotating cylindrical screen and is then brought into contact with a squeegee which acts to impel the heated ink composition through an array of apertures provided in the rotating cylindrical screen, the squeegee being fixedly disposed in the internal volume of the cylindrical screen so that the squeegee does not rotate within the cylindrical screen as the cylindrical screen is rotating;
contacting a printable substrate with an external surface of the rotating cylindrical screen to transfer a portion of the heated ink composition to the printable substrate through the apertures in the rotating cylindrical screen; and
cooling the portion of the ink composition on the printable substrate to a second predetermined temperature range, wherein the ink composition comprises a second viscosity range at the second predetermined temperature range, and wherein the second viscosity range is greater than the first viscosity range.

36. The method of claim 35, further comprising:
mixing the gelling agent and the carrier liquid to form the carrier composition at a third predetermined temperature range, wherein the third predetermined temperature range is greater than the first predetermined temperature range;
cooling the carrier composition to a temperature below the third predetermined temperature range; and
mixing the plurality of microcapsules into the carrier composition.

37. The method of claim 36, wherein the ink composition comprises a third viscosity range at the third predetermined temperature range, wherein the third viscosity range is less than the first viscosity range, and wherein the third predetermined temperature range is greater than the first predetermined temperature range.

38. (canceled)

39. The method of claim 35, wherein, after the portion of the ink composition on the printable substrate is cooled, the carrier composition of the portion of the ink composition on the printable substrate forms a gelled material comprises a network expanded throughout the carrier liquid.

40-52. (canceled)
